(19) United States
(12) Patent Application Publication
(10) Pub. No.: US 2007/0231856 A1
(43) Pub. Date: Oct. 4, 2007

(54) Recombinant Protein and Polypeptide Production Using Methylotropic or Ethylotropic Microorganisms with a Dilute Methanol or Ethanol Feeding

(76) Inventors: Pei Ye, Huntsville, AL (US); Daniel C. Carter, Huntsville, AL (US)

Correspondence Address: STITES & HARBISON PLLC, 1199 NORTH FAIRFAX STREET, SUITE 900, ALEXANDRIA, VA, 22314, US

(21) Appl. No.: 10/558,650

(22) PCT Filed: Jun. 1, 2004
(86) PCT No.: PCT/US04/17038

§ 371 Date: Feb. 23, 2007

Related U.S. Application Data
(60) Provisional application No. 60/474,235, filed on May 30, 2003.

(51) Int. Cl. C12N 5/06

(52) U.S. Cl. 435/69.1; 435/71.1

(57) ABSTRACT

A method is provided for expressing recombinant proteins and polypeptides such as human serum albumin (HSA), human growth hormone (HGH), or insulin-like growth factor-I (IGF-1) in large quantities using a methylotropic or ethylotropic microorganism such as a yeast, bacteria or fungi using a dilute methanol or ethanol feeding strategy which provides for a lower flash point and can minimize the likelihood of dangerous explosion. The present invention is thus advantageous because it allows recombinant proteins to be produced on a large scale in non-explosion proof plants without the hazards associated with the use of 100% methanol which generally Ces not meet OSHA requirements, and can thus allow the safe and efficient production of large quantities of recombinant proteins and other biomaterials at a far reduced cost.

### CROSS-REFERENCE TO RELATED APPLICATIONS

The present application claims the benefit of U.S. provisional application Ser. No. 60/474,235, filed May 30, 2003, the disclosure of which is incorporated herein as if set forth in its entirety including specification and drawings.

### FIELD OF THE INVENTION

The present invention relates in general to a method for expressing recombinant proteins using genetically engineered methylotropic or ethylotropic microorganisms such yeast, bacteria or fungi, and in particular to a system wherein such processes can be carried out using dilute methanol or ethanol feeding which has a lower flash point and which will allow large scale recombinant production using less expensive, non-explosion proof facilities so that large scale production of highly valuable proteins such as human serum albumin can be obtained at a fraction of the cost that would be involved if methanol or ethanol at high percentages were used in the production of the protein which would require elaborate and expensive explosion-proof facilities.

### BACKGROUND OF THE INVENTION

Genetic engineering processes utilizing methylotropic or ethylotropic microorganisms such as yeast, bacteria or fungi have long been known and used for the production of numerous valuable proteins and other products. In some of these systems, very efficient methylotropic and ethylotropic yeasts such as those from the genera Sacchromyces, Pichia, Hansenula, Candida, Kluyveromyces or Schwanniomyces are used to produce large scale quantities of recombinant proteins, and prokaryotic hosts, such as methylotropic bacteria such as E. coli, Bacillus and Streptomyces, can also be used. These recombinant proteins are generally manufactured using a fermentation process which uses a two stage feeding protocol. In such a process, cell mass is usually generated quickly by batch and/or feed-batch growth using glycerol as a carbon source. Once glycerol is depleted, a production phase is initiated by feeding with methanol or ethanol which induces a separate metabolic pathway which includes induction of the recombinant gene through the activation of a suitable promoter.

Exemplary of patents disclosing the use of methylotropic or ethylotropic yeast in the production of recombinant proteins or polypeptides are U.S. Pat. Nos. 5,330,901 and 5,707,828 (human serum albumin); 5,612,198 (insulin-like growth factor-I); 6,342,375 (human growth hormone); 5,965,389 (polypeptide GAD65); and 5,827,684 (Bacillus entomotoxins), all of said patents incorporated herein by reference.

In these microorganism processes, the action of methanol or ethanol is essential in order to obtain the high scale production of the desired recombinant protein. However, at present, such processes require use of methanol or ethanol at an extremely high percentage, e.g., 70% or higher, and at these extremely high percentages, methanol and ethanol are flammable and have a low flash point such that the danger of explosion is extremely high. As a result, it has previously only been possible to carry out such procedures using extremely expensive state-of-the-art recombinant facilities wherein very elaborate and expensive measures need to be taken to make the facilities explosion proof. Because of this limitation, many processes are only available at a large cost very often beyond the reach of many companies, and many facilities, including food grade manufacturing plants, which would otherwise be suitable to culture and produce recombinant proteins, cannot be used for this purpose.

One example of a protein that is highly desirable to be produced recombinantly and in large quantities is human serum albumin. Human serum albumin is a major protein of the circulatory system and plays an important role in numerous physiological functions as well, including a significant contribution to colloidal oncotic blood pressure (roughly 80%) and a major role in the transport and distribution of numerous exogenous and endogenous ligands. These ligands can vary widely and include chemically diverse molecules including fatty acids, amino acids, steroids, calcium, metals such as copper and zinc, and various pharmaceutical agents. Albumin generally facilitates transfer many of these ligands across organ-circulatory interfaces such as the liver, intestines, kidneys and the brain, and studies have suggested the existence of an albumin cell surface receptor. See, e.g., Schnitzer et al., P.N.A.S. 85:6773 (1988). Serum albumin generally comprises about 50% of the total blood component by dry weight, and is also chiefly responsible for controlling the physiological pH of blood. This protein is thus intimately involved in a wide range of circulatory and metabolic functions and vitally important not only to proper circulation and blood pressure but to the interactions and effects of pharmaceutical compositions when administered to a patient in need of such administration.

Human serum albumin (or “HSA”) is a protein of about 66,500 kD which is produced in the liver and is comprised of 585 amino acids including at least 17 disulphide bridges. As set forth above, it has an outstanding ability to bind and transport a wide spectrum of ligands throughout the circulatory system including the long-chain fatty acids which are otherwise insoluble in circulating plasma. The sequences and certain details regarding specific regions in albumin have previously been set forth, e.g., in U.S. Pat. No. 5,780,594 and U.S. Pat. No. 5,948,609, both of which are incorporated herein by reference. Other articles or references of relevance with regard to human serum albumin include Carter et al., Advances in Protein Chemistry, 45:153-203 (1994); Peters, Jr., “All About Albumin”, Academic Press (1995); Camerman et al., Can J. Chem., 54:1309-1316 (1976); Lau et al., J. Biol. Chem., 249:5878-5884 (1974); Callan et al., Res. Commun. Chem. Pathol. Pharmacol., 5:459-472 (1973); and Nieboer et al., Br. J. Ind. Med., 41:56-63 (1984); and all of these references are incorporated by reference as well. HSA is thus one of the major circulatory proteins, and because of its abundance in the circulatory system and its function as a carrier of various molecules, it is highly desirable because of its use in maintaining osmotic pressure of blood and its ability to be used in the determination of the safety and efficacy of many pharmaceuticals.

Like many other important blood proteins and other biological materials, HSA is usually administered clinically in large amounts, often over 10 grams per dose and thus tons of albumin purified from blood are administered annually worldwide. However, when using albumin purified from blood, the risk of transmission of pathogenic agents such as HIV, hepatitis viruses and possibly other, as yet unidentified viruses, is unacceptably high, and as a result, this has lead to great interest in producing recombinant HSA in a microorganism, such as yeast. Further, from a research aspect, HSA plays a very important role in cell culture performed by biotechnology companies, clinical and research labs. With a concern of potential contamination of pathogenic agents, more and more biotechnology companies, clinical and research labs have started replacing blood-derived HSA with recombinant HSA. Moreover, many other highly valuable recombinant proteins and other biomolecules as set forth above, including insulin growth factor-I and human growth hormone, can be produced recombinantly and which are designed for administration in human patients are in high demand for similar reasons and must be produced in large scale quantities.

This large demand means that a very large production capacity is needed. However, current production facilities worldwide do not have enough capacity to produce large amounts of recombinant proteins to fulfill the market demand. In addition, as indicated above, due to the involvement of flammable chemical such as methanol or ethanol during the production of recombinant proteins prepared using many yeast strains, most large classical fermentation facilities do not meet OSHA's safety requirement if adopting a high percentage (e.g., 100%) methanol or ethanol feeding method. Moreover, the safety issue is always a concern for such biological fermentation facilities.

Therefore, there is a distinct need in the field to provide a process which is capable of producing large amounts of recombinant proteins using the efficient methylotropic and ethylotropic microorganisms, and yet which can use a dilute methanol or ethanol feeding process so that the recombinant proteins can be produced safely and efficiently in plants that would otherwise not meet OSHA safety requirements.

### SUMMARY OF THE INVENTION

Accordingly, it is an object of the present invention to provide a method for producing recombinant proteins using methylotropic or ethylotropic microorganisms such as yeast, bacteria or fungi which does not require the presence of elaborate and very expensive explosion-proof facilities.

It is a further object of the present invention to provide a method of producing proteins recombinantly using yeast genera such as Sacchromyces, Pichia, Hansenula, Candida, Kluyveromyces or Schwanniomyces which can be carried out on a large scale at a greatly reduced cost.

It is yet further another object of the invention to provide a process for producing highly valuable recombinant proteins using microorganism systems requiring an alcohol such as methanol or ethanol as the carbon source and/or in the induction system using large, non explosion-proof facilities including those for food-grade manufacturing plants which currently cannot carry out such a procedure.

It is still another object of the present invention to provide a recombinant process which can utilize yeast promoters that are activated by an alcohol such as methanol or ethanol, and yet which can be carried out at a temperature below the flash point of the useful methanol/water and ethanol/water mixtures so as to meet the necessary federal, state or foreign flash point safety requirements in the location where the recombinant processes will take place.

It is even further an object of the present invention to provide a recombinant process which can utilize Pichia pastoris yeast at a diluted level of methanol or ethanol yet still produce recombinant proteins such as human serum albumin cheaply, safely and efficiently on a large scale

These and other objects are provided by virtue of the present invention which provides for the first time a method of producing recombinant proteins using a methylotropic or ethylotropic microorganism such as yeast, bacteria or fungi and only a diluted amount of methanol or ethanol so as to have a flash point lower than that required by OSHA and other health and safety regulations to prevent against dangerous explosions. In the preferred process, a methylotropic or ethylotropic yeast such as one from the genera Sacchromyces, Pichia, Hansenula, Candida, Kluyveromyces or Schwanniomyces, or a bacteria such as E. coli, Bacillus or Streptomyces is genetically engineered so as to include the target protein of interest, e.g., human serum albumin, and the process is carried out in a fermenter wherein there is an initial introduction of glycerol which is completely exhausted, followed by an introduction of dilute methanol or ethanol and a carbon source such as sorbitol. It is also preferred that the process utilize dilute methanol or ethanol in such an amount which ensures that the flash point, which is dependent on the level of methanol or ethanol dilution, is kept below that amount required by state, federal or foreign regulations concerning health and safety of manufacturing processes. The present process is extremely advantageous. In that it will allow many large scale non-explosion proof manufacturing plants, including food grade manufacturing plants, to be utilized in the production of highly valuable recombinant proteins, and this will allow the production of recombinant proteins in a far cheaper manner that before possible, and at the same time increase the profitability of the large scale manufacturing plants which now can be utilized to produce highly valuable recombinant proteins.

These embodiments and other alternatives and modifications within the spirit and scope of the disclosed invention will become readily apparent to those skilled in the art from reading the present specification and/or the references cited herein, all of which are incorporated by reference.

### DETAILED DESCRIPTION OF THE PREFERRED EMBODIMENTS

In accordance with the present invention, a process is provided wherein recombinant production of valuable proteins such as human serum albumin can be accomplished using a dilute methanol or ethanol feed system wherein the methanol or ethanol is kept below its flash point so as to remove the risk of explosion and allow for the large scale production of these proteins in food-grade, non-explosion proof facilities. In the preferred process, a methylotropic or ethylotropic yeast such as one from the genera Sacchromyces, Pichia, Hansenula, Candida, Kluyveromyces or Schwanniomyces, or a bacteria selected from E. coli, Bacillus or Streptomyces, is genetically engineered so as to include the target protein of interest, e.g., human serum albumin, and the process is carried out in a fermenter wherein there is an initial introduction of a sugar capable of supporting the growth of said microorganism, such as glycerol, which is completely exhausted, followed by an introduction of dilute methanol and a carbon source such as sorbitol. By dilute methanol or ethanol is meant an amount which allows a flash point which will be acceptable to satisfy health and safety requirements in the jurisdiction wherein the recombinant processes will take place. In general, although flash point may vary somewhat based on other conditions such as pressure and humidity, it is normally the case that methanol or ethanol percentage in the dilute formulations of the present invention will be no greater than about 30% methanol or ethanol by weight in aqueous medium. In that most State and Federal regulations require the Flash Points of mixtures in these large scale environments to be at or greater than about 100° F., it is particularly preferred that when the process uses dilute methanol, the aqueous methanol is approximately 26% or less (by weight), and when dilute ethanol is used, the aqueous level should be at 23% or less (by weight). The safety (Flash Point) requirements will vary with various state, federal or foreign regulations and can be adjusted accordingly, and thus any dilution of methanol or ethanol which will result in flash points which will satisfy said regulations will be suitable for use in the present invention and are contemplated as acceptable ranges in accordance with the invention. In general, the flash points of various dilutions of methanol and ethanol are known (see Tables 1 and 2), and thus the level of dilution of methanol or ethanol in accordance with the invention can be determined on the basis of what is the minimum flask point requirement for a given jurisdiction.

TABLE 1
Methanol/Water Mixtures

| | | |
|---|---|---|
| Methanol Conc. Wt. % | Freezing Point | Flash Point |
| (Vol. %) | F. (C.) | (TCC) F. (C.) |

| 0 | (0) | 32 | (0) | No Flash | |
|---|---|---|---|---|---|
| 10 | (13) | 20 | (−7) | 130 | (54) |
| 20 | (24) | 0 | (−18) | 110 | (43) |
| 30 | (35) | −15 | (−26) | 95 | (35) |
| 40 | (46) | −40 | (−40) | 85 | (29) |
| 50 | (56) | −65 | (−54) | 75 | (24) |
| 60 | (66) | −95 | (−71) | 70 | (21) |
| 70 | (75) | −215 | (<−73) | 60 | (16) |
| 80 | (83) | −225 | (<−73) | 55 | (13) |
| 90 | (92) | −230 | (<−73) | 55 | (13) |
| 100 | (100) | −145 | (<−73) | 55 | (13) |

TABLE 2
Ethanol/Water Mixtures

| | | |
|---|---|---|
| EtOH Conc. | Freezing Point, | Flash Point, |
| Vol. % (Wt.) | F. (C.) | (TCC) F. (C.) |

| 0 | (0) | 32 | (0) | No Flash | |
|---|---|---|---|---|---|
| 10 | (8) | 25 | (−4) | 135 | (57) |
| 20 | (17) | 15 | (−9) | 105 | (41) |
| 30 | (26) | 5 | (−15) | 90 | (32) |
| 40 | (34) | −10 | (−23) | 80 | (27) |
| 50 | (44) | −25 | (−32) | 80 | (27) |
| 60 | (54) | −35 | (−37) | 80 | (27) |
| 70 | (65) | *−55 | (−48) | 80 | (27) |
| 80 | (76) | *−75 | (−59) | 75 | (24) |
| 90 | (88) | *−110 | (<−73) | 65 | (18) |
| 100 | (100) | *−175 | (<−73) | 55 | (13) |

*Temperatures at which super cooling often occurs

In accordance with the present invention, the preferred process will utilize any suitable methylotropic or ethylotropic yeast which is commonly used in the recombinant preparation of proteins from genes inserted into the yeast. As would be recognized by one of ordinary skill in the art, such yeast are well known and include various members of yeast genera including Sacchromyces, Pichia, Hansenula, Candida, Kluyveromyces or Schwanniomyces. One particular yeast suitable in the present process is Pichia pastoris, and one particularly suitable strain is Pichia pastoris GS115. Heterologous protein expression in methylotropic and ethylotropic yeast using Pichia pastoris and other yeast has been known for a number of years, as described in Gregg et al., FEMS Microbiology Reviews 24:45-46 (2000), incorporated herein by reference. In the preferred process, prior to the culturing of the recombinant yeast, the initial step is to genetically engineer the yeast strain so as to be able to express the desired protein using suitable promoters well known to those skilled in the art.

In general, the preferred process, the primary modification over the conventional processes used to culture methylotropic and ethylotropic microorganisms is the use of the diluted and augmented feed source featuring methanol or ethanol at a percentage low enough to keep the flash point low so as to avoid the possibility of explosion that will be present using conventional yeast culturing processes that have used a high percentage (e.g., 100% methanol or ethanol), yet still be able to induce expression of the recombinant protein or polypeptide. In the current processes, the main purpose is to use more expensive sugars capable of supporting microorganism growth, e.g., glycerol, on a more limited basis in a step-wise process wherein optical density (“OD”) of the cultured cells reaches an initial desired level. After sufficient cell density is achieved, a less expensive sugar is used to continue building cell density and volume (the greater the cell density at the time of induction, the more efficient the recombinant production). At the desired cell density in the main fermentor (which will vary with protein and yeast strain), the medium is allowed to deplete the initial carbon source so as to switch carbon sources (metabolic pathways) to allow for the recombinant expression/production. In accordance with the present invention, a second carbon source is added which constitutes a carbon source that uses a metabolic pathway that does not interfere with the methanol induction. In other words, a second carbon source is used which does not result in repressing transcription of the recombinant protein induced by the methanol or ethanol. In one particularly preferred embodiment, this second carbon source which does not interfere with methanol induction is sorbitol, but many other similar sugars can be used as the second carbon source provided that their metabolic pathways do not interfere with methanol induction. The added sorbitol allows for cell growth and mass to continue at desirable rates while allowing for the production of the recombinant protein to take place by virtue of the diluted methanol which induces protein expression. In the present invention, it is preferred that while the initial step may use glycerol or other “normal” sugars which can support microorganism growth and thus start building up cell density, such sugars would interfere with methanol induction, and thus in accordance with the invention, it is desired that such initial sugars be consumed prior to the step of dilute methanol or ethanol induction.

In the preferred process, pre-prepared inoculums of the desired recombinant microorganisms are obtained as described herein, and are prepared for culturing in a suitable fermenter in any of a number of desirable ways. In the normal process, inoculums for the dilute methanol process of the invention can be prepared by taking a vial of the host cell frozen stock and inoculated it into a suitable amount (e.g., 50 ml) of an appropriate culture medium, e.g., MGY medium in a 300 ml baffled flask. The culture is then grown at a suitable temperature for promoting cell growth (e.g., 28-30° C.) in a shaking incubator (at speeds of generally 250-300 rpm) for a desired period, e.g., overnight. Next, following the initial growth of the culture, a small amount (e.g., 3 ml) of the culture is used to inoculate a suitable amount of a culture medium (e.g., 300 ml of MGY medium) in an appropriate container (e.g., 2 L baffled flask), and this is once again grown at a suitable temperature (e.g., 28-30° C.) in the shaking incubator (250-300 rpm preferred) until the culture reaches a suitable optical density, e.g., OD₆₀₀=10. As indicated above, the recombinant yeast may be genetically engineered to express any suitable protein or polypeptide that can be expressed in said system, and these proteins and polypeptides can include the serum albumins such as human serum albumin, human growth hormone, insulin-like growth factor-1, polypeptide GAD65, Bacillus entomotoxins, etc. With regard to human serum albumin, a recombinant albumin having genetic modifications such as disclosed in PCT application WO 02/05645 may be used, said patent publication incorporated herein by reference. Other information concerning recombinant production of human serum albumin can be found in Quirk et al., “Production of Recombinant Human Serum Albumin”, Biotech and Applied Biochemistry, pg. 273-289 (1989), said article incorporated herein by reference.

Following preparation of the inoculums as set forth above, the fermentation process is carried out which generally comprises inoculating with the previously prepared inoculums as described above a culture medium containing a first carbon source capable of supporting growth of the culture medium such as glycerol, glucose, fructose, sucrose and mixtures thereof and then subjecting the culture medium to conditions of temperature, agitation, air flow and pH so as to maintain levels of dissolved oxygen sufficient to promote the growth of the microorganisms in the inoculum; proceeding to culture the inoculum in the medium until the first carbon source is consumed; and then optionally shifting the culture to a second carbon source such as sorbitol whose metabolic pathways do not interfere with the methanol or ethanol induction while introducing dilute methanol or ethanol in sufficient quantifies to induce the expression of the desired recombinant protein whose genes have been inserted into the methylotropic or ethylotropic microorganism as set forth above. Following the desired level of culturing in the fermentor, the desired protein or polypeptide may be isolated, purified, and/or recovered from the culture medium. As would be recognized by one of ordinary skill in this art, the particular desired parameters with regard to pH, dissolved oxygen, agitation, air flow, temperature control and feed rates will vary based on the protein being produced, and will be well known details to the skilled artisan based on the desired requirements of the given protein or polypeptide being recombinantly produced.

In accordance with the invention, a suitable process for producing recombinant proteins from genetically engineered microorganism may be carried out on a small scale or large scale depending on the nature and purpose of the process. For example, it may be desirable to carry out a test run on a small scale to obtain routine information concerning the desired parameters for a particular recombinant protein before conducting large scale processes. However, in accordance with the invention, it is highly desirable to utilize the present invention in large scale manufacturing facilities to produce the desired recombinant proteins or polypeptides, and because of the use of dilute methanol or ethanol as set forth herein, such plants will generally meet OSHA and other local health and safety requirements even though they will normally not contain highly complex and expensive explosion-proof measures.

In the processes described below, yeast is used as the methylotropic or ethylotropic microorganism, however, as would be recognized to one skilled in the art, these processes may be utilized for other microorganisms such as bacteria and fungi with appropriate modifications as would be known to one skilled in the art. In addition, it is recognized that a protease-deficient strain such as yeast strains KM71, SMD 1163, SMD 1168 may also be used.

In one suitable process in accordance with the invention, a feed-batch fermentation using a dilute methanol feeding may be carried out using a suitable fermenter, and for small-scale operations, a 15 L BioFlo 110 fermenter may be used. In this process, the 15 L fermenter having a working volume of 10.5 L and containing 3 L of glycerol medium (initial concentration 5% w/v) may be inoculated with 300 ml of inoculum. The temperature, pH, and DO can be set at 30° C., 5.0, and 30%, respectively, for the glycerol batch and 25° C., 5.85, and 30%, respectively, for the methanol feed-batch, with the pH being controlled using a 30% NH₄OH solution (v/v). In accordance with the invention, the glycerol is completely exhausted, at which point the OD₆₀₀ may be about 100. Following exhaustion of the glycerol, an initial dilute methanol feed-batch phase is initiated. In this step, the dilute methanol feed rate is started at 1 mL (100%)/L.h and increased over 6-8 h to 6 mL/L.h. As indicated above, since most State and Federal regulations require the Flash Points of mixtures in these large scale environments to be at or less than 100° F., use of a dilute aqueous methanol mixtures at approximately 26% or less would generally be considered safe under those criteria, and for aqueous ethanol, the dilute level that would generally be considered safe would be about 23% or less. As also indicated above, the safety (Flash Point) requirements will vary with various state, federal or foreign regulations and thus the level of methanol or ethanol dilution may be adjusted accordingly. The feeding pump setting depends on the dilution of methanol (1-99%), and a feeding rate of about 6 mL/L.h should be suitable and may be maintained during the length of the fermentation or adjusted accordingly based on DO spike performance. The final OD₆₀₀ may reach 250 or higher.

Following the initial methanol induction process as referred to above, a continuous fermentation process containing a restarting of the dilute methanol induction may take place. In such a system, after an initial recovery of e.g., 7.5 L of the fermenter sample from initial fermentation cycle, the dilute methanol induction process may be restarted and continued until a desired level of recombinant protein is obtained. In this process, total yields of the recombinant protein or polypeptide using the dilute methanol in accordance with the invention are compatible to the yield that would be produced using 100% methanol feeding in a similar time frame.

In the preferred process of the present invention, methylotropic or ethylotropic yeast may be used in large processes in order to obtain high volumes of a desirable recombinant protein or polypeptide. In such large scale operations, the present invention can be used to produce a yield of recombinant product on a par with conventional 100% methanol systems, but yet which utilized a safer dilute methanol feeding strategy. In carrying out such processes, any suitable yeast strain as set forth above which can be genetically engineered to express a desired protein or polypeptide may be utilized in accordance with the invention. For a large scale operation, a suitable large fermenter, such as a 10,000 L fermenter may be employed. If a 10,000 L fermenter is used, the following volumes and conditions may be used, and other volumes can similarly be used wherein the volumes and conditions set forth below would be adjusted to scale. In the preferred process, the fermenter with a working volume of about 10,000 L containing about 3500 L of glycerol medium is inoculated with about 12 L ml of yeast inoculums as described above. In this process, temperature, agitation, air flow and pH are maintained at a level which will be conducive to growth of the yeast cells and which will be conducive to maintaining sufficient dissolved oxygen (DO) to allow the process to go forward. For example, temperature may be set at around 29° C. for batch and glycerol feed and 25° C. for the induction phase wherein methanol or ethanol will be introduced along with a second carbon source that will have a metabolic pathway that does not interfere with the methanol induction. It is preferred that agitation be set at approximately 50-200 RPM and cascaded to DO control, and that air flow be set at approximately 225-470 SCFM and cascaded to DO control. The pH may be controlled by a number of suitable means, e.g., ammonia, and it is desirable to have the pH be maintained at about 5.0 for the batch and glycerol feeds and about 5.85 for the dilute methanol or ethanol induction phase. It is also preferred that DO be maintained at greater than 20%, e.g., by either increasing agitation in 50 RPM increments or increasing air flow in 50 SCFM increments.

As indicated above, the initial steps are carried out until consumption of glycerol in the batch medium, which is followed by the dilute methanol or ethanol induction accompanied by the introduction of a second carbon source, namely one whose metabolic pathways do not interfere with methanol or ethanol induction. One such suitable second carbon source will be sorbitol, but one skilled in the art will recognize that many such carbon sources can be utilized in the invention provided that they do not interfere with methanol or ethanol induction. As the process continues and cell mass reaches a suitable amount, the feed rate may be adjusted as necessary based on the methanol and sorbitol concentration in the culture broth. The duration of the feed phase is dependent upon OTR of the vessel and working volume of the vessel, and a total duration of about 50 hr±0.5 hr may be suitable to obtain sufficient amounts of the desired recombinant product.

Accordingly, using the dilute methanol or ethanol feeding strategy in accordance with the present invention, large scale production of highly desirable recombinant proteins and peptides may be obtained wherein the yields are comparable to the yield of the recombinant products produced using 100% methanol or ethanol feeding in a similar time frame. However, the present invention has the distinct advantage of being able to be utilized in large scale manufacturing plants, including those prepared for food grade level manufacturing, which do not have explosion-proof facilities and which would otherwise be unable to conduct recombinant processes using methylotropic or ethylotropic yeast.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Small Scale Test Run

A small scale test run of the dilute methanol feeding strategy of the present invention was conducted using a Pichia pastoris yeast strain genetically engineered to express human serum albumin. In the general process, inoculums of the recombinant yeast are fermented in a process having a glycerol source which is allowed to be completely exhausted before the introduction of the dilute methanol feed.

Inoculum Preparation

Inoculums for the dilute methanol process of the invention were prepared as follows:

a. One vial of the host cell frozen stock from cell bank was inoculated to 50 ml of MGY medium in a 300 ml baffled flask. The culture is grown at 28-30° C. in a shaking incubator (250-300 rpm) overnight.

b. Following initial growth of the culture, 3 ml of the culture is used to inoculate 300 ml of MGY in a 2 L baffled flask and grown at 28-30° C. in a shaking incubator (250-300 rpm) until the culture reaches OD₆₀₀=10.

Fermentation

A feed-batch fermentation using a dilute methanol feeding was carried out in accordance with the invention using a 15 L BioFlo 110 fermenter under the following conditions:

a. The 15 L fermenter (working volume 10.5 L) containing 3 L of glycerol medium was inoculated with 300 ml of inoculum.

b. The initial glycerol concentration was 5% (w/v).

c. The temperature, pH, ad DO were set at 30° C., 5.0, and 30%, respectively, for glycerol batch and 25° C., 5.85, and 30%, respectively, for methanol fed-batch.

d. The pH was controlled using a 30% NH₄OH solution (v/v).

e. When the glycerol was completely exhausted, the OD₆₀₀ was about 100.

f. Initial dilute methanol feed-batch phase is initiated. In this step, the dilute methanol feed rate is started at 1 mL (100%)/L.h and increased over 6-8 h to 6 mL/L.h. Since most State and Federal regulations require the Flash Points of mixtures in these large scale environments to be at or less than 100 F, so this means that aqueous methanol mixtures at approximately 26% or less would be considered safe and for aqueous ethanol at 23% or less. The safety (Flash Point) requirements will vary with various state, federal or foreign regulations and can be adjusted accordingly. The feeding pump setting depends on the dilution of methanol (1-99%). The feeding rate of 6 mL/L.h was maintained during the length of the fermentation or adjusted accordingly based on DO spike performance. The final OD₈₀₀ may reach 250 or higher.

g. Continuous fermentation. After recovery 7.5 L of the fermenter sample from initial fermentation cycle, the dilute methanol induction process was restarted. Using this process, several cycles of rHSA production were accomplished with a total rHSA yield compatible to the yield of rHSA produced with 100% methanol feeding in a similar time frame. The feeding pump setting depends on the dilution of methanol (1-99%). The feeding rate of 6 mL/L.h was maintained during the length of the fermentation or adjusted accordingly based on DO spike performance. The final OD₆₀₀ may reach 250 or higher.

### Example 2

### Large Scale Commercial Level Production

The process of the present invention was then used in a large scale run which evidenced the successful use of the process in providing a yield of recombinant product on a par with conventional 100% methanol systems, but yet which utilized a safer dilute methanol feeding strategy.

In the initial steps of the process, the strain of the yeast Pichia pastoris GS115 was genetically engineered to express human serum albumin, and then inoculums are prepared as stated in Example 1.

Fermentation

The fermentation is carried out in a 10,000 L fermenter under the following conditions:

a. The fermenter (working volume 10,000 L) containing 3500 L of glycerol medium was inoculated with 12 L ml of inoculums.

b. The temperature is set at 29° C. for batch and glycerol feed and 25° C. for Methanol-Sorbitol mix-feed.

c. Agitation is set AT 50-200 RPM and cascaded to DO control.

d. Air flow is set at 225-470 SCFM and cascaded to DO control.

e. The pH was controlled using Ammonia, with a maintenance of pH 5.0 for the batch and glycerol feeds and 5.85 for the Methanol-Sorbitol mix-feed.

f. DO is maintained at greater than 20% by either increasing agitation in 50 RPM increments or increasing air flow in 50 SCFM increments.

g. After glycerol consumption in the batch medium, the cell mass concentration reaches 25 g/L. Glycerol feed is started when OUR peaks or DO spikes.

h. Methanol-Sorbitol mix-feed is started when cell mass reaches 46 g/L. The feed rate is adjusted based on the methanol and sorbitol concentration in the culture broth. The duration (50 hr±0.5 hr) of the feed phase is dependent upon OTR of the vessel and working volume of the vessel. The cell mass concentration reaches 78 g/L.

Productivity

Taking into account of cell contribution, cell volume and supernatant volume, yields are calculated based on the cell biomass. The yield of rHSA was determined using BCG assays and SDS-PAGE. The yield of rHSA using the dilute methanol process of the present invention was comparable to the yield of rHSA produced using 100% methanol feeding in a similar time frame.

What is claimed is:

1. A method for the recombinant production of proteins or polypeptides from a methylotropic or ethylotropic microorganism using a dilute methanol or ethanol feed comprising:
a. inoculating a methylotropic or ethylotropic microorganism which has been genetically engineered to express a recombinant protein or polypeptide when induced with methanol or ethanol, respectively, into a culture medium containing a carbon source capable of promoting the growth of the inoculated microorganism;
b. maintaining the dissolved oxygen in the culture medium at a level which will promote the growth of the inoculated microorganism;
c. growing or culturing the microorganism in the culture medium until the carbon source is consumed; and
d. adding dilute methanol or ethanol in an amount sufficient to induce the expression of the recombinant protein or polypeptide either alone or along with a second carbon source which does not result in repressing transcription of the recombinant protein induced by the methanol or ethanol.

2. The method of claim 1 wherein the recombinant protein, polypeptide, cell mass or effluent is isolated, purified, or recovered from the culture medium.

3. The method of claim 1 wherein the methylotropic or ethylotropic microorganism is selected from the group consisting of yeast, bacteria and fungi.

4. The method of claim 3 wherein the methylotropic or ethylotropic microorganism is a yeast of a genus selected from the group consisting of Sacchromyces, Pichia, Hansenula, Candida, Kluyveromyces or Schwanniomyces.

5. The method of claim 3 wherein the methylotropic or ethylotropic microorganism is a bacteria selected from the group consisting of E. coli, Bacillus and Streptomyces.

6. The method of claim 4 wherein the ethylotropic yeast is Kluyveromyces lactis.

7. The method of claim 6 wherein the methylotropic or ethylotropic yeast is from strain Pichia pastoris GS115.

8. The method of claim 1 wherein the dissolved oxygen is maintained at a level of about 10% or greater.

9. The method of claim 1 wherein the dissolved oxygen is controlled by either increasing agitation, air flow or oxygen content during the fermentation process.

10. The method of claim 1 wherein the dilute methanol or ethanol comprises no greater than 30% methanol or ethanol by weight in an aqueous medium.

11. The method of claim 1 wherein the methanol or ethanol is at a dilution sufficient to ensure that the flash point will be about 100° F. or greater.

12. The method of claim 1 wherein a methylotropic yeast is used, and wherein the induction is carried out using dilute methanol comprising no greater than about 26% by weight in an aqueous medium.

13. The method of claim 1 wherein an ethylotropic yeast is used, and wherein the induction is carried out using dilute ethanol comprising no greater than about 23% by weight in an aqueous medium.

14. The method of claim 1 wherein the carbon source capable of supporting growth of the inoculated microorganisms is selected from the group consisting of glycerol, glucose, fructose, sucrose and mixtures thereof.

15. The method of claim 1 wherein the carbon source which does not result in repressing transcription of the recombinant protein induced by the methanol or ethanol is selected from the group consisting of sorbitol, lactose, and mixtures thereof.

16. The method of claim 1 wherein the recombinant protein or peptide is selected from the group consisting of serum albumin, human growth hormone, insulin-like growth factor-1, polypeptide GAD65 and Bacillus entomotoxins.

17. The method of claim 1 wherein the recombinant protein is human serum albumin.

18. The method of claim 1 wherein the method is carried out in a non-explosion proof facility.

19. A method for the recombinant production of proteins or polypeptides from a methylotropic or ethylotropic yeast using a dilute methanol or ethanol feed comprising:
a. inoculating a methylotropic or ethylotropic yeast which has been genetically engineered to express a recombinant protein or polypeptide when induced with methanol or ethanol, respectively, into a culture medium containing glycerol;
b. maintaining the dissolved oxygen in the culture medium at a level which will promote the growth of the inoculated yeast;
c. growing or culturing the yeast in the culture medium until the carbon source is consumed; and
d. adding dilute methanol or ethanol in an amount sufficient to induce the expression of the recombinant protein or polypeptide along with a carbon source which does not result in repressing transcription of the recombinant protein induced by the methanol or ethanol.

20. The method of claim 18 wherein the initial glycerol concentration is 5%+/−(w/v).

21. A method for the recombinant production of proteins or polypeptides from a methylotropic or ethylotropic yeast using a dilute methanol or ethanol feed comprising:
a. inoculating a methylotropic or ethylotropic yeast which has been genetically engineered to express a recombinant protein or polypeptide when induced with methanol or ethanol, respectively, into a culture medium containing glycerol;
b. maintaining the dissolved oxygen in the culture medium at a level which will promote the growth of the inoculated yeast;
c. growing or culturing the yeast in the culture medium until the carbon source is consumed; and
d. adding dilute methanol or ethanol in an amount sufficient to induce the expression of the recombinant protein or polypeptide along with sorbitol in an amount that will promote the growth of the inoculated yeast.

22. The method of claim 19 wherein the feed rate is adjusted based on the concentration of the methanol or ethanol and sorbitol in the culture medium.

23. A method according to claim 1 wherein the microorganism is a protease deficient strain.

24. A method according to claim 23 wherein the microorganism is a yeast selected from the group consisting of KM71, SMD 1163, SMD 1168.
